SEQUENCE LISTING (2 sequences)

SEQ ID NO: 1 - protein, 12 aa, Pseudomonas stutzeri
NPSSLFRYLPSD

SEQ ID NO: 2 - protein, 12 aa, Pseudomonas stutzeri
AYSSGAPPMPPF

(19) United States
(12) Patent Application Publication
(10) Pub. No.: US 2013/0123466 A1
(43) Pub. Date: May 16, 2013

(54) SIMULTANEOUS SYNTHESIS OF TEMPERATURE-TUNABLE PEPTIDE AND GOLD NANOPARTICLE HYBRID SPHERES

(71) Applicants: Hor-Gil HUR, Buk-gu (KR); Jungok Kim, Buk-gu (KR)

(72) Inventors: Hor-Gil HUR, Buk-gu (KR); Jungok Kim, Buk-gu (KR)

(73) Assignee: GWANGJU INSTITUTE OF SCIENCE AND TECHNOLOGY, Buk-gu (KR)

(21) Appl. No.: 13/295,815

(22) Filed: Nov. 14, 2011

(51) Int. Cl. C07K 17/14; C07K 1/04; B82Y 15/00

(52) U.S. Cl. 530/345; 530/300; 977/810

(57) ABSTRACT

The present invention relates to a novel synthesis of peptide-gold nanoparticle hybrid spheres comprising a step of forming a hybrid structure by inducing self-assembly of a gold-binding peptide, and forming a gold nanoparticle in the structure at the same time. According to the present invention, size of the structure can be controlled according to temperature, and it can be used for various biomedical and electronic applications using the structure.

### TECHNICAL FIELD

The present disclosure relates to a synthesis of peptide-gold nanoparticle hybrid spheres and the peptide-gold nanoparticle hybrid spheres produced thereby.

### BACKGROUND

Self-assembled biological molecules (e.g., peptides, proteins, DNA, etc.) have been investigated as excellent templates to assist the formation of hierarchical inorganic nanoengineered materials in environmental benign ways, while typical self-assembly of biological molecules occurs via non-covalent bonding, electrostatic attraction, hydrogen bonding, hydrophobic bonding and aromatic stacking interactions (1, 2). Moreover, self-assembled biomaterials which can change their shapes, conformations and physical properties in response to environmental variables (e.x., temperature, pH, light) have a potential being applicable in biomedical and bionanotechnology applications in drug delivery, bio-sensing and tissue engineering (3-6). Although various stimuli-responsive organisms are formed by synthetic polymers and peptides, particularly, peptides can be directly separated from microorganism or indirectly separated by phase separation and a cell surface expression method. Further, peptide is one of the most attractive materials because it is able to 1) control its chemical properties by adjusting primary amino acid sequence, 2) chelate to comprise various inorganic ions and reduce them to form metallic, semiconducting and insulating inorganic nanostructures (7-11), and 3) control the crystal structure and morphology by adjusting the nucleation and growth mode of inorganic nanomaterials, under moderate environment (7, 12-17). In addition, some peptides are able to self-assemble so as to form, for example, complex structures including chains, sheets, and spheres (18-22).

Most of these heterostructures were synthesized using a two-step process of the self-assembly of peptide to form the scaffolds/templates followed by synthesis of inorganic nanostructures.

More recently, a single-step process was developed that allows for the simultaneous formation of structural complex and highly ordered nanostructures. For example, a self-assembling cationic diphenylalanine peptide, which was previously known to be useful for the synthesis of peptide nanotubes and nanospheres, can be directly used to the synthesis of spherical, peptide-polyoxoanion (phosphotungstic acid, PTA) hybrid nanostructures in water (23-26). In addition, an aliphatic carbon-tailed peptide AG3 (AYSSGAPPMPPF), which has high binding affinity to silver, can produce double-helical structures in HEPES buffer (22). Despite these advances, there are still limited problems on the assembly of biological molecules of stimulus-response and well-designed supramolecular structures under aqueous conditions.

In this study, the present inventors explain a simple one-step method to form peptide-gold nanoparticle hybrid spheres at physiological temperature in an aqueous solution. A peptide of SEQ ID NO: 1 (NPSSLFRYLPSD), which was isolated using a bateriophage-displayed combinatorial peptide library, was previously used to synthesize peptide-gold nanoparticle hybrid spheres in an aqueous solution (9).

Once the spheres were formed, removal of the gold nanoparticles from the hybrid structures has no influence on the safety of the spheres. In addition, it was shown that size of the peptide-gold nanoparticle hybrid spheres could be controlled in a temperature-dependent manner. This method, which combines peptide-based supramolecular structures with desirable functional inorganic materials, may provide a novel method for using development of “bottom-up” fabrication of hierarchical structures with unique physical, chemical and biological properties.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY

The present inventors have made intensive researches to develop a simple method, particularly one-step method to produce peptide-gold nanoparticle hybrid spheres. As a result, the present inventors have found that self-assembly of a gold-binding peptide is induced, and a scaffold to a hybrid structures is formed as soon as gold nanoparticles are formed in the scaffold when the gold nanoparticles are produced using a gold ion reductant and a gold-binding peptide as a self assembly conductor.

Accordingly, an object of the present invention is to provide a synthesis of peptide-gold nanoparticle hybrid spheres.

Another object of the present invention is to provide peptide-gold nanoparticle hybrid spheres.

Other objects and advantages of the present invention will become apparent from the following detailed description together with the appended claims and drawings.

In one aspect of the present invention, provided is a synthesis of peptide-gold nanoparticle hybrid spheres comprising a step of contacting (i) a gold ion reductant and a gold-binding peptide as a self-assembly inducer with (ii) a gold salt to induce self-assembly of the gold-binding peptide, and forming a scaffold to a hybrid structure as soon as forming a gold nanoparticle in the scaffold.

In another aspect of the present invention, provided is peptide-gold nanoparticle hybrid spheres produced by the said method.

The present inventors have made intensive researches to develop a simple method, particularly one-step method to produce peptide-gold nanoparticle hybrid spheres. As a result, the present inventors have found that self-assembly of a gold-binding peptide is induced, and a scaffold to a hybrid structures is formed as soon as gold nanoparticles are formed in the scaffold when the gold nanoparticles are produced using a gold ion reductant and a gold-binding peptide as a self assembly conductor.

### BRIEF DESCRIPTION OF THE DRAWINGS

The application file contains drawings executed in color (FIGS. 2, 8, 9 and 11). Copies of this patent or patent application with color drawings will be provided by the Office upon request and payment of the necessary fee.

FIGS. 1a to 1d are images and a graph of a self-assembled peptide and a gold nanoparticle hybrid sphere, and FIG. 1a is a SEM image, FIG. 1b is a graph of EDX analysis, and FIGS. 1c and 1d are TEM images.

FIGS. 2a to 2c are data for cross-sectioned analyses of a self-assembled peptide and a gold nanoparticle hybrid sphere, and FIG. 2a is a TEM image, FIG. 2b is HR-TEM images and FIG. 2c is EDX mapping data.

FIGS. 3a to 3c are a graph showing the reduction of the gold ion concentration in a time-dependent manner by the peptide of SEQ ID NO: 1 (FIG. 3a) and TEM images obtained from the 3 hour incubation (FIGS. 3b and 3c).

FIG. 4a is a TEM image of self-assembled peptides and gold nanoparticle hybrid spheres whose size is controlled according to temperature (above) and a graph showing size distribution (below) at 37° C.

FIG. 4b is a TEM image of self-assembled peptides and gold nanoparticle hybrid spheres whose size is controlled according to temperature (above) and a graph showing size distribution (below) at 50° C.

FIG. 4c is a TEM image of self-assembled peptides and gold nanoparticle hybrid spheres whose size is controlled according to temperature (above) and a graph showing size distribution (below) at 70° C.

FIGS. 5a and 5b are TEM images for the effect of gold etching by KI/I₂ on a self-assembled peptide and a gold nanoparticle hybrid sphere.

FIGS. 6a to 6d are TEM images for the functional analyses of peptide sequences on self-assembled peptides and gold nanoparticle hybrid spheres by mutated peptides Y to G (FIG. 6a), Y to S (FIG. 6b), F to G (FIG. 6c) and FY to GS (FIG. 6d).

FIG. 7 is a schematic diagram showing the formation of a self-assembled peptide and a gold nanoparticle hybrid sphere.

FIG. 8 is a graph analyzing CD spectrum for peptide of SEQ ID NO: 1 with gold (red line) and without gold (black line).

FIGS. 9a to 9c are SEM images (FIGS. 9a and 9b) and EDX analysis (FIG. 9c) of a self-assembled peptide and a gold nanoparticle hybrid sphere after gold etching treatment with KI/I₂.

FIGS. 10a to 10d are TEM images showing stability of a self-assembled peptide and a gold nanoparticle hybrid sphere with treatment of protease K under acidic and basic conditions.

FIG. 11 is confocal laser scanning microscope (CLSM) images of peptide FAM-Si#6-C bounded to a self-assembled peptide and a gold nanoparticle hybrid sphere.

### DETAILED DESCRIPTION

The gold-binding peptide used in the present invention works as a gold ion reductant and self-assembly inducer. The gold-binding peptide as a gold ion reductant is bound to the gold ion and then reduced the gold ion. The gold-binding peptide as a self-assembly inducer induces self-assembly by interaction between R-groups of amino acids making up the gold-binding peptide.

As used herein, the term “peptide” refers to a linear molecule formed by linking amino acid residues through peptide bonds.

The peptide of the present invention can be prepared in accordance with chemical synthetic methods well known in the art, particularly, solid-phase synthesis techniques (Merrifield, J. Amer. Chem. Soc. 85:2149-54 (1963); Stewart, et al., Solid Phase Peptide Synthesis, 2nd. ed., Pierce Chem. Co.: Rockford, 111 (1984)).

Further, the peptide of the present invention can be obtained by a phage display technology (Smith G P “Filamentous fusion phage: novel expression vectors that display cloned antigens on the virion surface”. Science 228(4705):13151317 (1985); Smith G P, Petrenko V A. “Phage display”. Chem. Rev. 97(2):391410 (1997)). Further, the peptide of the present invention may be prepared in accordance with gene cloning methods. More specifically, the nucleotide sequences coding for the gold-binding peptide are transformed into suitable host cells and expressed to produce the gold-binding peptide (see Sambrook, J. et al., Molecular Cloning. A Laboratory Manual, 3rd ed. Cold Spring Harbor Press (2001)).

According to a preferred embodiment of the present invention, the gold-binding peptide used in the present invention comprises an amino acid having an aromatic functional group as R-group (for example, Trp, Tyr or Phe). More preferably, the gold-binding peptide comprises at least 2 amino acids having an aromatic functional group as R-group.

As proved in Example, the amino acid having an aromatic functional group plays very important role in the formation of peptide-gold nanoparticle hybrid spheres.

The length of the gold-binding peptide used in the present invention is not particularly limited, and the peptide consists of 10-20 amino acid residues, preferably, and 10-15 amino acid residues, more preferably.

According to a preferred embodiment of the present invention, a peptide of the gold-binding peptide comprises the amino acid sequence selected from the group consisting of SEQ ID NOs: 1 and 2, and more preferably, SEQ ID NO: 1.

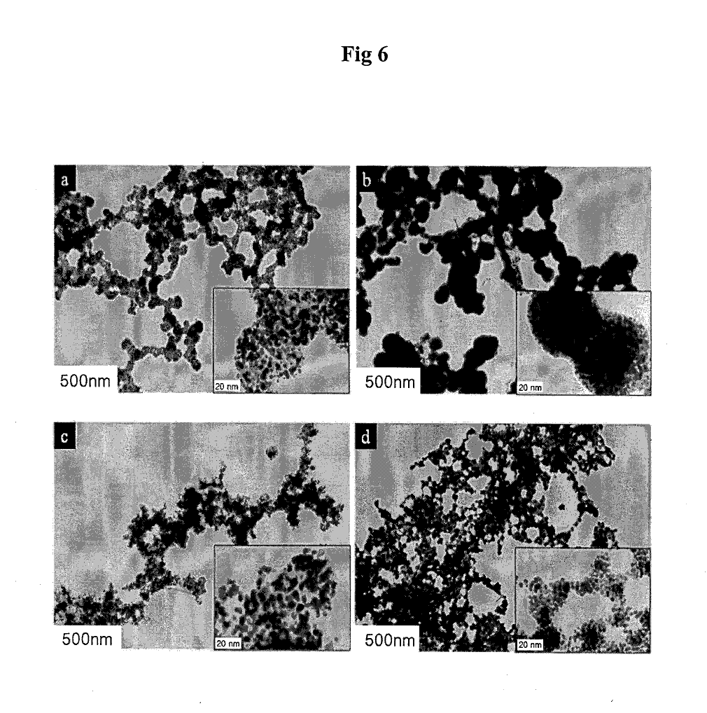

As demonstrated and illustrated in Example, to determine the relationship between the primary sequence of the peptide and the peptide-gold nanoparticle hybrid spheres, when phenylalanine and tyrosine residues in the sequence of the peptide of SEQ ID NO: 1, which have an aromatic functional group, are substituted with glycine and serine residue, respectively, the peptide sphere structures are not formed, and instead, networked, wire-like, non-patterned structure are produced. In Example, the peptide of SEQ ID NO: 1 is substituted, and the peptide sequences used are NPSSLFRSLPSD (Y to S), NPSSLFRGLPSD (Y to G), NPSSLGRYLPSD (F to G) and NPSSLGRSLPSD (F and Y to G and S, respectively). Results in FIG. 6 show that any of the amino acid substitutions in the peptide of SEQ ID NO: 1 could not form the peptide sphere structures. It is considered that the spheres are formed by the interaction (for example, non-covalent bonding, electrostatic attraction, hydrogen bonding, hydrophobic bonding or aromatic stacking interaction) between an amino acid having an aromatic functional group and an amino acid having other R-group in a peptide, and the interaction between amino acids having an aromatic functional group of identical peptide molecules.

According to a preferred embodiment of the present invention, the gold salt used in the present invention includes any gold salt used to produce gold particles. The gold particles can be obtained by simply reacting the gold salt and the gold-binding peptide without any help of other materials. Preferred gold salts include HAuCl₄, HAuBr₄, NaAuCl₄, AuCl₃.3H₂O and NaAuCl₄.2H₂O, and most preferably, HAuCl₄. HAuCl₄ (Chloroauric acid) is dissociated to square planar [AuCl₄] ion and proton in aqueous condition, and plays a role as a precursor to in consisting a gold-coordinate complex.

According to a preferred embodiment of the present invention, the concentration of the gold salt ranges from 0.01 to 1.0 mM, preferably, from 0.05 to 0.2 mM, more preferably, and from 0.08 to 0.15 mM, most preferably.

According to a preferred embodiment of the present invention, the inventive peptide-gold nanoparticle hybrid spheres are formed in aqueous solution. Nanoparticles can be synthesized in an organic solvent and the like by various physicochemical methods, but those methods have problems of high energy consumption, high cost and high toxicity. The present invention is a more improved invention than the existing synthesis because it can introduce the particles in a manner which is environmental-friendly and non-toxic to a human body when the reaction is conducted at living body-like condition such as aqueous condition.

According to a preferred embodiment of the present invention, the pH of the reaction with the gold salt is 1.0-5.0, preferably, and 3.0-4.0, more preferably. In basic condition of high pH, the stability of the peptide-gold nanoparticle hybrid spheres decreased, and therefore, the conformation could not be maintained and finally, dissociated. Further, because the spheres are made of peptides, it is difficult to maintain the conformation thereof due to the decreased stability at environment wherein protease such as protease K is added thereto. The reaction time is 1-48 hours, preferably, 12-36 hours, more preferably, and about 24 hours, most preferably.

Meanwhile, the present invention provides a method to control size of the peptide-gold nanoparticle hybrid spheres according to temperature. For example, when the peptide-gold nanoparticle hybrid spheres were produced at various temperatures in accordance with the present invention, average diameter of the peptide spheres was reduced by approximately 56% and 76%, respectively when the temperature increased from 37° C. (473 nm) to 50° C. (208 nm) and 70° C. (114 nm).

Besides the temperature recorded in Example, regarding to diameter size control of the peptide-gold nanoparticle hybrid spheres, the spheres can be produced by using characteristics that the diameter size becomes small with increased temperature in consideration of binding of the spheres to a receptor which requires size-dependent specificity and size to pass through a membrane channel. Particularly, it is found that the size range of the produced spheres becomes consistent with increased temperature, and therefore, the technical advantages of the present invention can be applicable to areas requiring the size consistency.

According to a preferred embodiment of the present invention, size distribution of the peptide-gold nanoparticle hybrid spheres becomes more consistent with increased temperature.

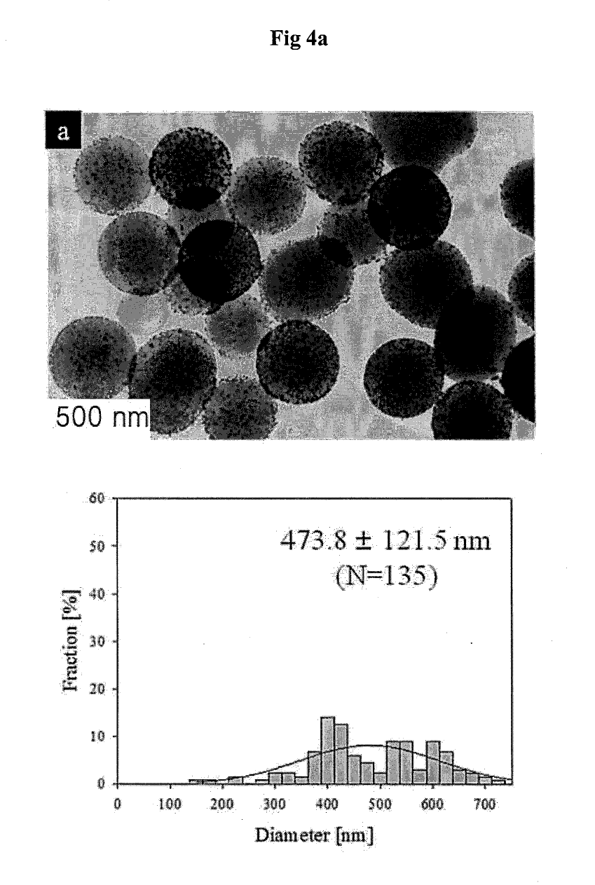

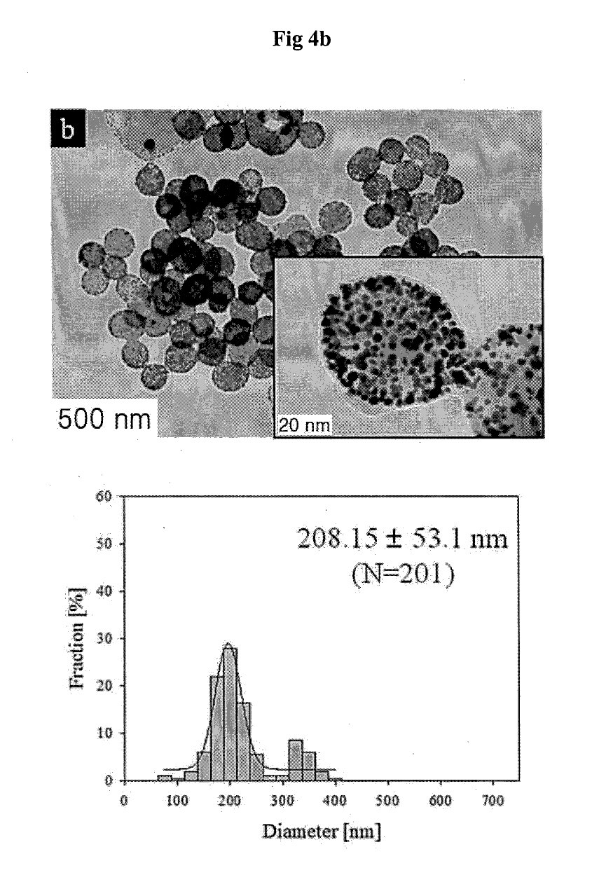

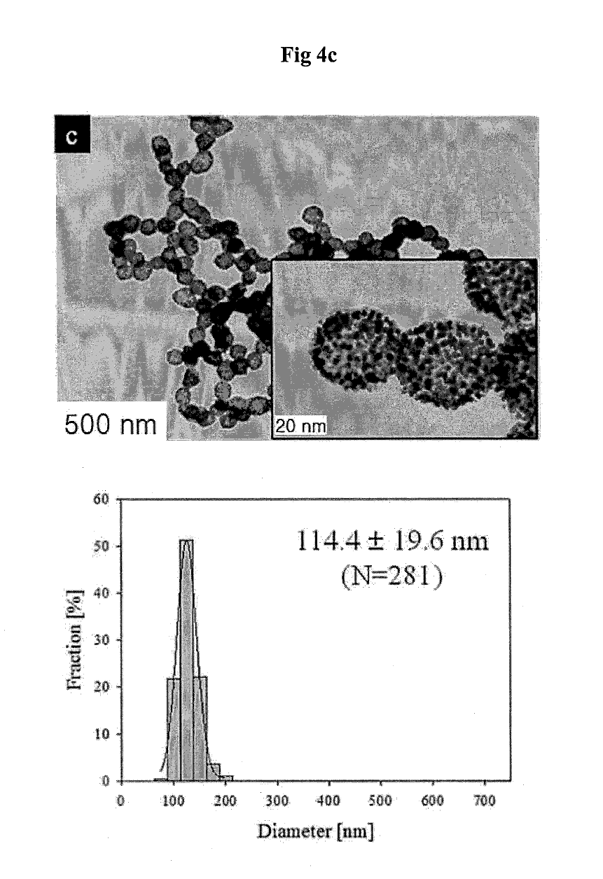

In FIGS. 4a to 4c, size distribution of the peptide-gold nanoparticle hybrid spheres becomes more consistent with increased temperature (473.8±121.5 nm (FIG. 4a), 208.15±53.1 nm (FIG. 4b) and 114.4±19.6 nm (FIG. 4c)).

The inventive peptide-gold nanoparticle hybrid spheres have own uses (for example, CT contrast medium or thermal therapy), but further functional materials can be bounded thereto to give more functionalities.

According to a preferred embodiment of the present invention, the method of the present invention further comprises a step of functionalizing the surface of the peptide-gold nanoparticle hybrid spheres. The term “functionalization” used herein refers to conjugating an optional functional material to the surface of the spheres by the peptide-gold nanoparticle hybrid spheres.

The functional material which can be conjugated by the peptide-gold nanoparticle hybrid spheres is not particularly limited, and, for example, include a medicament and a marker. Further, the functional material includes nucleic acid molecules (DNA, RNA), proteins, peptides, lipids, carbohydrates and low molecular compounds.

A maker can be conjugated by the peptide-gold nanoparticle hybrid spheres includes any marker known in the art, and, for example, FAM™, TAMRA™, HEX™, fluorescein, rhodamine, lucifer yellow, B-phycoerythrin, 9-Acridine isothiocyanate, lucifer yellow VS, 4-acetamido-4′-isothiocyanatostilbene-2,2′-disulfonic acid, 7-diethylamino-3-(4′-isothiocyanatophenyl)-4-methylcoumarin, succinimidyl-pyrene butyrate, 4-acetamido-4′-isothiocyanatostilbene-2,2′-disulfonic acid derivative, LC™-Red 640, LC™-Red 705, Cy5, Cy5.5, lysamine, isothiocyanate, erythrosin isothiocyanate, diethylenetriamine pentacetate, 1-dimethylaminonaphthyl-5-sulfonate, 1-anilino-8-naphthalene sulfonate, 2-p-touidinyl-6-naphthalene sulfonate, 3-phenyl-7-isocyanatocoumarin, 9-isothiocyanatoacridine, acridine orange, N-(p-(2-benzoxazolyl)phenyl)maleimide, benzoxadiazole, stilbene and pyrene, and derivatives thereof.

The functionalization of the surface of the peptide-gold nanoparticle hybrid spheres can be performed using various methods known in the art. For example, a functional material combined with various reacting groups can be conjugated to the surface of the peptide-gold nanoparticle hybrid spheres. The reacting groups used for conjugation may include aldehyde; epoxy; haloalkyl; primary amine; thiol; maleimide; ester (preferably, N-hydroxysuccinimide ester functional group); and reacting groups, which can be activated, such as carboxyl group (activated by the formation of hydroxy-succinimide ester) and hydroxyl group (activated by cyanogens bromide), but not limited thereto.

The functionalization of the surface of the peptide-gold nanoparticle hybrid spheres can be performed by covalently bonding the functional materials to a peptide terminal end of the spheres or gold, preferably, gold.

The peptide-gold nanoparticle hybrid spheres of the present invention can be used in various areas such as drug delivery system, contrast medium and bionanotechnology.

### Example

Hereinafter, the present invention will be more particularly described by the preferred examples. However, these are intended to illustrate the invention as preferred embodiments of the present invention and do not limit the scope of the present invention.

Materials and Methods

Chemical Reagents and Peptides

HAuCl₄.3H₂O and ClAuPMe₃ were purchased from Aldrich Chemicals (St. Louis, Mo.), and HNO₃ was purchased from Duksan Pure Chemicals Co. Ltd (Korea). Nanopure water used was prepared by the Milli-Q system (Millipore, Billerica, Mass.) and autoclaved prior to use to avoid microbial contamination. All other chemical reagents were reagent grade. All peptides used were purchased from Any Gen Co. Ltd. (Gwangju, Korea).

Simultaneous Synthesis of Peptide-Gold Nanoparticle Hybrid Spheres

Peptide of SEQ ID NO: 1 (0.2 mg) and HAuCl₄.3H₂O were added in a final reaction volume of 1 ml (pH about 3), followed by incubation at 37° C. in the dark for 24 hrs. Mutated peptides, with specific amino acid changes in the peptide sequence, were functionally analyzed using the same reaction conditions as described above. The influence of temperature on the synthesis of gold nanoparticles with peptide spheres was determined by incubating the reaction mixture at 50° C. or 70° C. The synthesized nanomaterials were collected by centrifugation at 9,300 g for 5 min at 25° C., washed twice with autoclaved deionized water, and then resuspended in 100 μL of water for further analyses.

Circular Dichroism (CD) Spectroscopy

The CD spectra of nanostructures were recorded from 190 to 250 nm by using a J-810 spectropolarimeter (Jasco, Tokyo, Japan) at 298 K and a quartz cuvette with 1 mm path length. Analyses were performed using 0.2 mg/mL of peptide in water after 24 h of incubation at 37° C. under dark conditions.

Kinetic Studies

The change in gold ion concentration with time was examined by three times of an experiment using reaction samples consisting of 0.2 mg of peptide of SEQ ID NO: 1 and 0.1 mM of HAuCl₄. The samples were incubated at 37° C., and centrifuged after 0, 1, 3, 6, 12 or 24 hrs. The resulting supernatant solutions were diluted with 2% HNO₃, and the gold ion concentration was measured by inductively coupled plasma optical emission spectrometry (ICP-OES) using an Optima model 5300DV instrument (PerkinElmer, Waltham, Mass.).

Gold Etching

The synthesized gold nanoparticles were removed from the surface of the peptide spheres by treatment with KI/I₂ gold etching solution. KI/I₂ gold etching solution was prepared by the addition of 4 g of KI, 1 g of I₂ and 40 ml of water with constant stirring at 25° C. 20 ml of etching solution was added to 100 ml of washed sample. The mixture was incubated for 5 min at 25° C. and washed two times with deionized water.

Stability Test

Stability of the peptide-gold nanoparticle hybrid sphere structures was measured after incubations at acidic and basic conditions and catalyst treatment. The procedure of the previous study (1) was repeated except for treating 10% trifluoroacetic acid or 1 M NaOH to 100 ml sample of the synthesized peptide-gold nanospheres for 5 hrs at room temperature. For catatlyst reaction condition, the nanospheres were treated with 20 mg of protease K (GeneAll Biotechnology, Seoul, Korea) in Tris-HCl buffer (pH 8.0) and incubated for 1 hr at 37° C. After the incubation, the sample was centrifugated, washed with deionized water and resuspended before analysis.

Surface Functionalization with Fluorescent Dye-Labeled Peptide

The assembled peptide and gold nanoparticle hybrid spheres were incubated with 1 mg of FAM-Si#6-C peptide for 24 h at 20° C. The samples were centrifuged and washed twice with deionized water. The intensity of the bound fluorescent-dye-labeled peptide with the gold nanoparticles on the hybrid spheres was analyzed by using confocal laser scanning microscopy (CLSM, LSM5, Zeiss, Germany).

Structural Characteristics of Synthesized Nanomaterials

The formed nanostructures were characterized by a field emission transmission electron microscopy (FE-TEM), high-resolution TEM (HR-TEM), scanning electron microscopy (SEM), and energy-dispersive X-ray spectroscopy (EDX). TEM analysis was conducted using a Tecnai F20 FE-TEM (Philips Electron Optics, Eindhoven, The Netherlands), and EDX analysis was conducted using a JEM-2100 HR-TEM (JEOL, Tokyo, Japan) at an accelerating voltage of 200 kV. The samples were prepared by depositing the nanostructures onto carbon-coated Cu support grids followed by air drying. The samples for SEM analyses were analyzed at an accelerating voltage of 10 kV by using a Hitachi S-4700 SEM (Tokyo, Japan) prepared for EDX analysis. The samples were prepared by placing 5 μl of a suspension containing nanostructures on a silicon wafer followed by air drying.

Results and Discussion

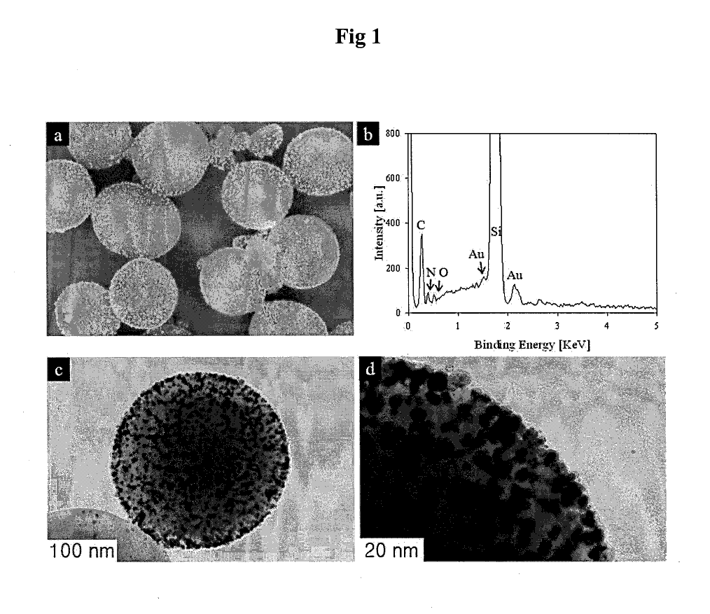

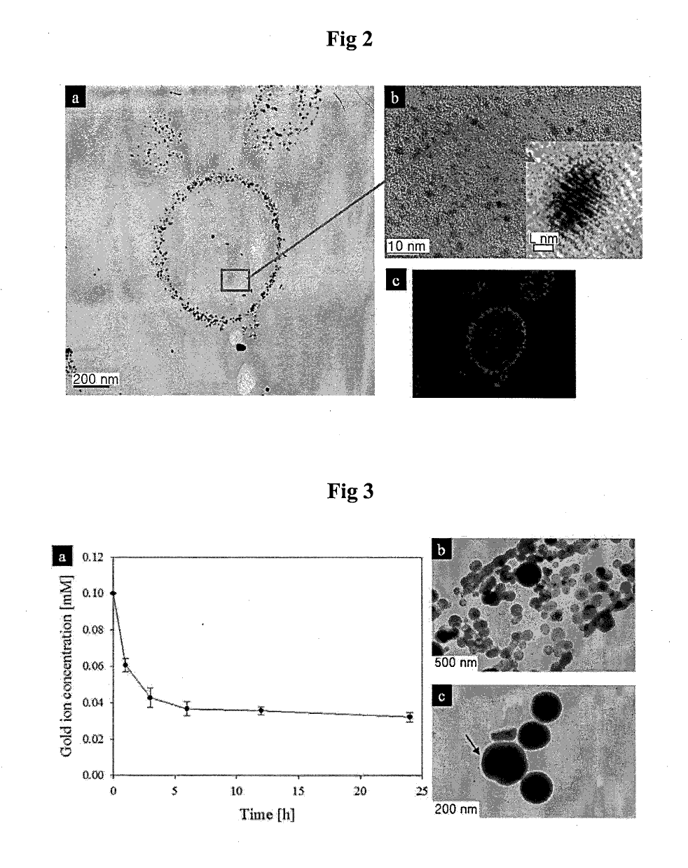

The peptide of SEQ ID NO: 1 was used as a precursor simultaneously to form peptide spheres and to reduce gold ions to gold nanoparticles under aqueous condition at physiological temperatures. The peptide of SEQ ID NO: 1, which was isolated using a bateriophage-displayed combinatorial peptide library, was previously shown to have high affinity to silver and be able to reduce silver ions to metallic silver nanoparticles in phosphate buffer at room temperature (9). When the peptide of SEQ ID NO: 1 (0.2 mg) was incubated in 0.1 mM HAuCl₄ for 24 hrs at 37° C., dark and violet-colored precipitates were formed, and it indicated that the gold nanoparticles were reduced by the peptides. TEM and SEM analyses indicated the formation of submicrometer spherical hybrid of peptide-gold nanoparticle conjugates with an average diameter of 473.8 nm (FIG. 1). EDX analysis confirmed that the submicrometer spherical structures contained gold nanoparticles (FIG. 1b). Incubation of chloroauric acid in the absence of the peptide of SEQ ID NO: 1 did not result in the formation of any precipitates or sphere-like structures. Similarly, no violet-colored precipitates and sphere structures were formed when water-insoluble organic gold (ClAuPMe₃) was incubated with the peptide of SEQ ID NO: 1 under the same reaction conditions (data not shown). This indicated that chloroauric acid appears to play an important role in the self-assembly of the peptide of SEQ ID NO: 1 and the nucleation and growth of gold nanoparticles within the peptide structures. Whereas the analysis of the TEM image shown in FIG. 1d indicated that the surface-associated gold nanoparticles had an average size of 8.6 nm, whether the location of gold nanoparticles on the surface or within the peptide spheres was undefined by these analyses. Cross-sectional analysis was performed to determine the distribution of the gold nanoparticles within these spherical hybrid structures. Results of cross-sectional TEM image analyses (FIG. 2a) clearly showed that the larger gold nanoparticles (average diameter of 8.6 nm) were predominately located on the surface of the peptide sphere, whereas results of HR-TEM analysis indicated that smaller gold nanoparticles (average diameter ranging from 2.5 to 4.5 nm) were located inside of the hybrid sphere (FIG. 2b). EDX electron microprobe analysis of gold confirmed the presence of gold nanoparticles throughout the spherical peptide structures (FIG. 2c). These results indicate that the peptide of SEQ ID NO: 1 can self-assemable to form submicrometer spherical particles that contain metallic gold nanoparticles and simultaneously reduce gold ions. It is considered that the small-sized gold nanoparticles were formed during initial reduction by the peptide of SEQ ID NO: 1 embedded inside of the peptide spheres. Meanwhile, the surface of the gold nanoparticles is likely to be in the continuous growth process with exposing them to the gold ions in the reaction solution. This was proved by the fact that the gold nanoparticles (average diameter of 8.6 nm) of the surface of the peptide sphere were larger than the gold nanoparticles (average diameter ranging from 2.5 to 4.5 nm) which were found inside of the hybrid sphere. Kinetic studies were performed to determine the reaction rate of gold ions as a function of incubation time. ICP-OES analysis (FIG. 3a) showed that gold ion concentration decreased from 0.1 to 0.04 mM after 6 hrs of incubation, followed by the slight reduction of gold ion concentration to 0.03 mM after 24 hrs of incubation. These results indicated that the peptide of SEQ ID NO: 1 was involved in the rapid reduction of the gold ions during early reaction stages. TEM images were taken to examine intermediate structures of the peptide spheres that interact with the gold nanoparticles in the early stages of reaction. Results in FIG. 3b show that neck-like structures and small-sized peptide spheres (90 to 400 nm diameter) were formed during the first 3 hrs of reaction (5). In addition, results in FIG. 3c show that hemisphere structures and incomplete peptide spheres, with different internal structures, are present and that small peptide spheres are likely incorporated into larger-sized ones via agglomeration.

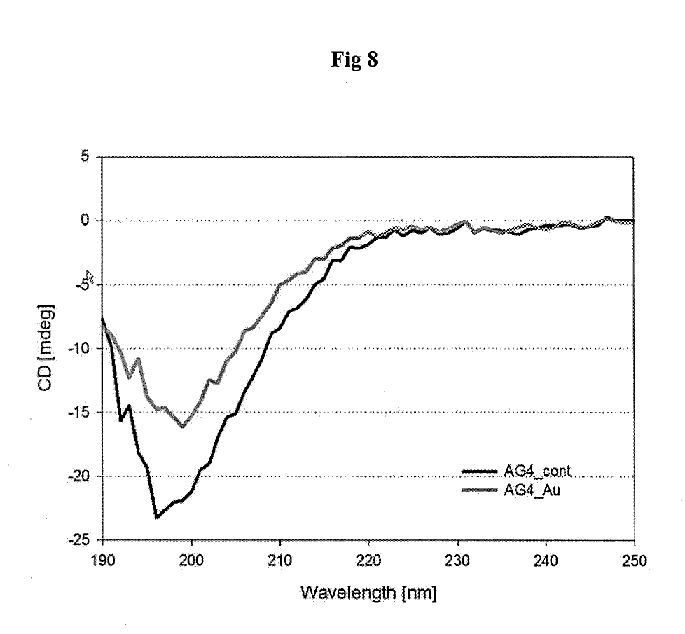

The secondary structure of the peptide of SEQ ID NO: 1 was examined using CD spectrum. The peptide of SEQ ID NO: 1 in the presence of the gold ion showed no ordered secondary structure but a random coil conformation of the peptide in water indicated by a strong negative peak at 199 nm, and it showed lower intensity that that of control experiment at the condition of the absence of the gold ion in a solution (FIG. 8). This result suggests that the peptide of SEQ ID NO: 1 possesses the random coil conformation regardless of the presence of the gold ions. However, the presence of the gold ions causes a decrease in the intensity of CD signal, and it is related to the conformation of peptide sphere structures with specific interaction with the gold ions (27). Results in FIG. 4 showed that size of the peptide spheres is controlled by reaction temperature. Average diameter of the peptide spheres were reduced by approximately 56% and 76%, respectively when the temperature increased from 37° C. (473 nm) to 50° C. (208 nm) and 70° C. (114 nm) (FIG. 4). However, despite the reduction in the size of the peptide spheres, the size of the gold nanoparticles distributed on the surface of the spheres were not significantly changed in response to the reaction temperature, and average diameter of the gold nanoparticles was 8.6 nm at 37° C., 8.3 nm at 50° C. and 8.1 nm at 70° C. (FIG. 4).

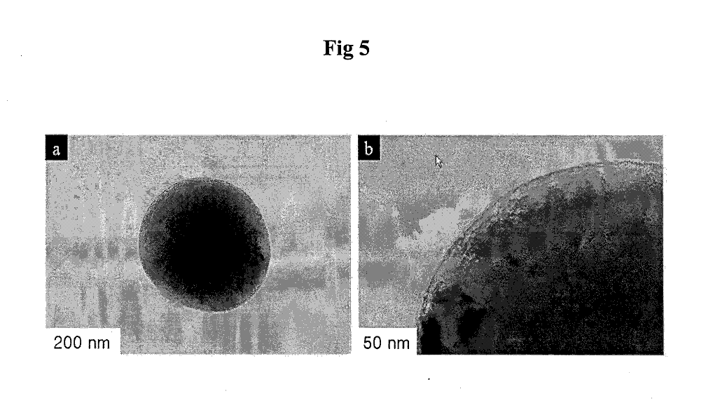

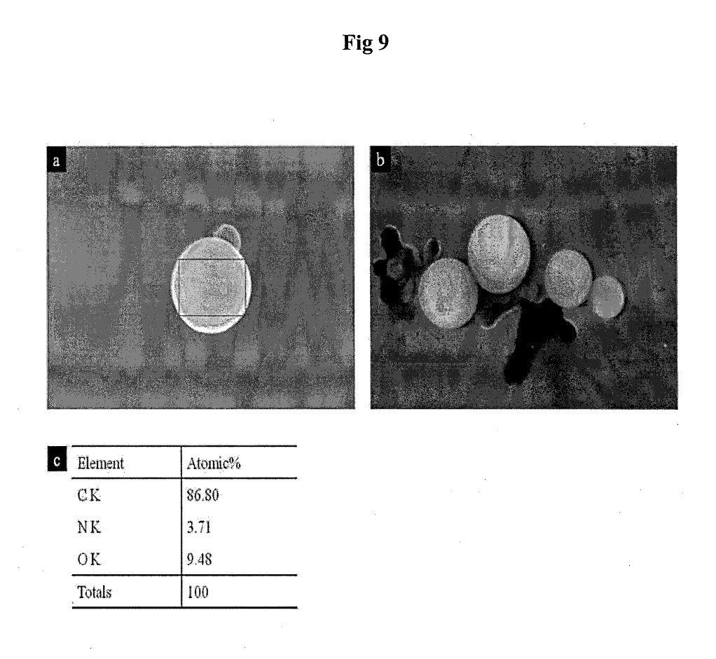

To obtain peptide spheres in the absence of the gold nanoparticles, the gold nanoparticles were dissolved on the surface and inside of the peptide sphere structures by treatment with a KI/I₂ solution. EDX analysis confirmed the absence of elemental gold nanoparticles inside and on the surface of the peptide sphere structures after KI/I₂ treatment (FIG. 9). The TEM images in FIG. 5 show that KI/I₂ etching resulted in the successful removal of the gold nanoparticles from the sphere structures. In addition, these studies showed that the peptide spheres were stably maintained in the absence of gold nanoparticles. High-resolution TEM images also show that the surface of the peptide spheres without the gold nanoparticles in contrast with the ragged surface of the hybrid structures with embedded gold nanoparticles (FIGS. 1 and 5b). Taken together, these results indicate that, once the self-assembled peptide sphere structures are formed, they remain stable without being affected by the gold nanoparticles. In addition, not only heterogeneous structures of the peptide and the gold nanoparticle hybrid but also homogeneous self-assembled peptide spheres without the gold nanoparticles may provide extended potential applications such as antibacterial agents or gene and drug delivery system (28, 29).

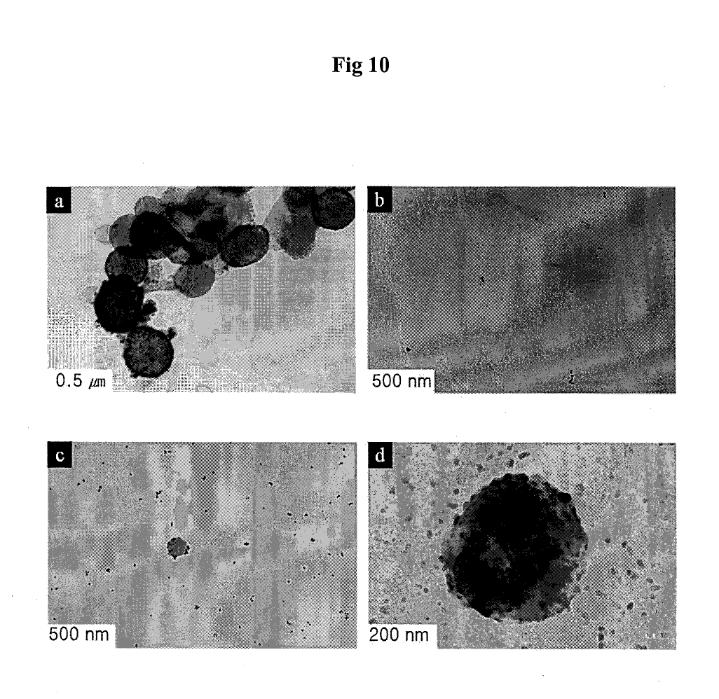

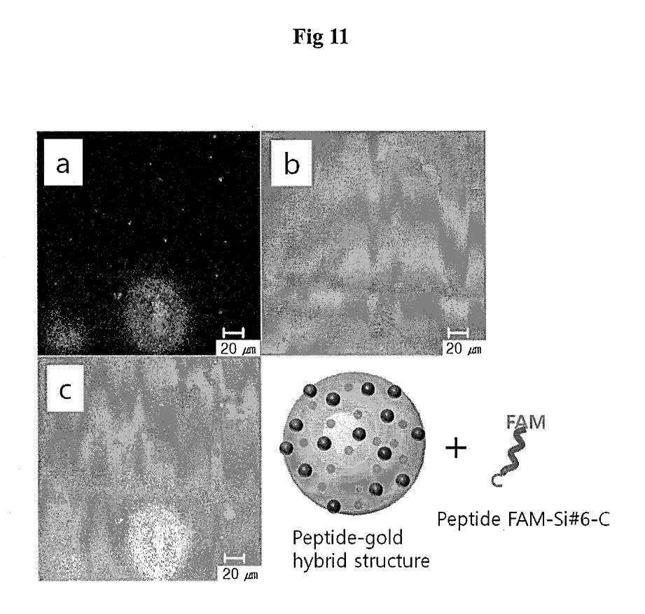

The stability of the hybrid sphere should be observed under extreme chemical and catalystic condition for future applications' of bionanotechnology (30). The self-assembled peptide and gold nanoparticle hybrid spheres were found to be stable at 25° C. after 10% TFA treatment (FIG. 10a). In addition, the peptide spheres maintained after removing the gold nanoparticles by KI/I₂ treatment were stable under very acidic condition formed by 10% TFA treatment (FIG. 10d). In contrast, the peptide-gold nanoparticle hybrid spheres were disassembled after incubation thereof for 5 hrs under basic condition (1 M NaOH) (FIG. 10b). Similarly, the hybrid spheres were degraded and the gold nanoparticles were released therefrom after incubation thereof for 1 hr at 37° C. with protease K treatment (FIG. 10c). These results show that the self-assembled peptide spheres are stable regardless of the presence of the gold nanoparticle under acidic condition, but unstable under basic condition or in the presence of protease. This means that the hybrid spheres can provide specific uses as a novel functional structure under acidic condition.

To determine the relationship between the primary sequence of the peptide of SEQ ID NO: 1 and the peptide-gold nanoparticle hybrid spheres, phenylalanine and tyrosine residues in the sequence of the peptide of SEQ ID NO: 1, which have an aromatic functional group, were substituted with glycine and serine, respectively. The peptide sequences used in these analyses were NPSSLFRSLPSD (Y to S), NPSSLFRGLPSD (Y to G), NPSSLGRYLPSD (F to G) and NPSSLGRSLPSD (F and Y to G and S, respectively). Results in FIG. 6 show that any of the tested amino acid substitutions in the peptide of SEQ ID NO: 1 could not form the peptide sphere structures. Instead, all of the mutated peptides produced networked, wire-like, non-patterned aggregates of peptides with the gold nanoparticles (FIG. 6). Interestingly, the gold nanoparticles were still produced by the mutated peptides, even when tyrosine, which acts as an active reductant for gold, was replaced with glycine or serine (31). Results of these studies demonstrated that the tyrosine residue alone is not sufficient for the reduction of gold ions, and several single amino acid changes within the peptide of SEQ ID NO: 1 inhibit the self-assembly of the nanosphere structures. Moreover, the primary structure of the peptide of SEQ ID NO: 1 appears to have a novel function that directs the synthesis of the nanosphere structures in the presence of the gold ions. It should be noted that it was previously shown that changes in the internal amino acids in some peptides affect the synthesis, shape and aggregation of the gold nanostructures (10). For example, substitution of one of the tyrosine residues with serine in peptide A3 resulted in the loss of the ability to form the gold nanoparticles and self-assembed peptide nanoribbons with the gold nanoparticles (22, 32). Despite this knowledge, the causal relationship between the primary structures of the peptide of SEQ ID NO: 1 and the sphere formation is currently unknown.

The surface functionalization of the gold nanoparticle hybrid spheres was exploited by using a previously designed peptide (FAM-Si#6-C), which links the fluorescent dye NHS-fluorescein to the N-terminal end and the C-terminal end linked to cysteine, respectively (FIG. 11). This peptide was previously shown to bind gold. Confocal laser scanning microscopy was used to measure the intensity of light emitted by the fluorescent-dye labeled peptide that is specifically bound to the gold nanoparticles in the hybrid structures. Results shown in FIG. 11 indicate that the FAM-Si#6-C peptide binds onto the surface of the gold nanoparticles containing hybrid spheres. This suggests that the gold nanoparticles exposed on the surface of the hybrid structures can be easily modified with bioactive molecules containing thiol (—SH) groups and that these hybrid structures may be useful for a variety of applications.

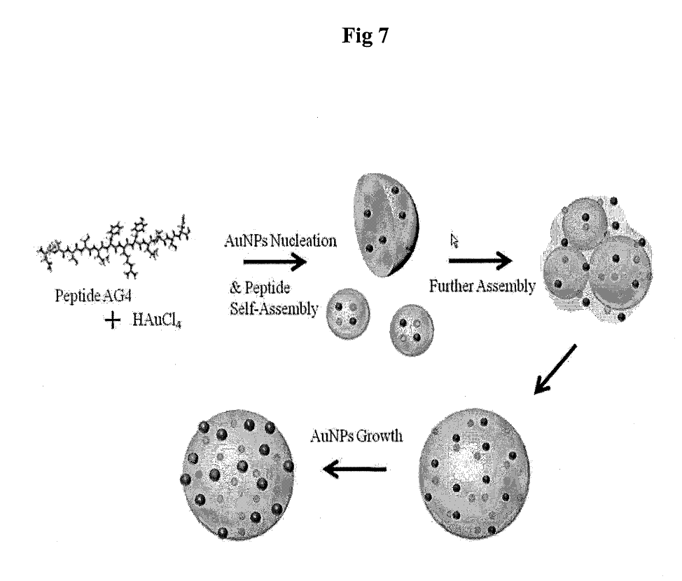

The diagram of FIG. 7 shows a synthesis of self-assembled peptide-gold nanoparticle hybrid spheres. The self-assembly of the peptides and the nucleation of the gold ion are occurs at the same time, and it is assisted by the specific interaction of the peptide of SEQ ID NO: 1 and chloroauric acid. The early reaction stages of the assembly process are proceeded via neck-like structures and small-sized peptide spheres, hemisphere structures and incomplete peptide spheres. These spheres are aggregated and grow together with the gold nanoparticles localized both inside and on the surface of the peptide sphere to form the peptide-gold nanoparticle hybrid spheres.

The present inventors have demonstrated the fabrication of temperature-tunable, the peptide and the inorganic hybrid spherical structures composed of the self-assembled peptide with the gold nanoparticles. The hybrid spheres were formed under aqueous acidic conditions at 37° C. The gold nanoparticles that formed in this reaction were localized both inside and on the surface of the peptide sphere, and it indicates that the simultaneous self-assembly of the peptides and nucleation of the gold ion via the specific interaction of both reactants. The size of the peptide spheres with the gold nanoparticles was tunable and inversely related to reaction temperature. The gold removal experiment showed that once the peptide spheres were formed, they were stable regardless of the presence of the gold nanoparticles. Assembly of the spheres was dependent on the primary amino acid sequence of the peptide and was specifically influenced by amino acids containing aromatic functional groups. The method used here to form multifunctional hybrid biomolecules may be useful to produce various nanostructures that may be widely used to applications in the biomedical, electronic and nanotechnological areas for the production of drug delivery, bioimaging, and biosensor systems.

The summary of features and advantages of this invention is as follows:
(a) The present invention provides a method to produce peptide-gold nanoparticle hybrid spheres by one-step.
(b) The present invention provides a method to control the size of the peptide-gold nanoparticle hybrid spheres according to temperature.
(c) The synthesized spheres whose size is controlled according to the present invention can be used to various applications such as nanospheres and electronic microscope required for biomedical or biological systems.

While certain embodiments have been described, these embodiments have been presented by way of example only, and are not intended to limit the scope of the disclosures. Indeed, the novel methods and apparatuses described herein may be embodied in a variety of other forms; furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

### REFERENCES

1) Lim, Y.-B.; Moon, K.-S.; Lee, M. Chem. Soc. Rev. 2009, 38, 925-934.
2) Gazit, E. Chem. Soc. Rev. 2007, 36, 1263-1269.
3) Mart, R. J.; Osborne, R. D.; Stevens, M. M.; Ulijn, R. V. Soft Matter 2006, 2, 822-835.
4) Lowik, D. W. P. M.; Leunissen, E. H. P.; van den Heuvel, M.; Hansen, M. B.; van Hest, J. C. M. Chem. Soc. Rev. 2010, 39, 3394-3412.
5) Yan, X.; Zhu, P.; Li, J. Chem. Soc. Rev. 2010, 39, 1877-1890.
6) Kim, E.; Kim, D.; Jung, H.; Lee, J.; Paul, S.; Selvapalam, N.; Yang, Y.; Lim, N.; Park, C. G.; Kim, K. Angew. Chem.-Int. Edit. 2010, 49, 4405-4408.
7) Dickerson, M. B.; Sandhage, K. H.; Naik, R. R. Chem. Rev. 2008, 108, 4935-4978.
8) Sarikaya, M.; Tamerler, C.; Jen, A. K.-Y.; Schulten, K.; Baneyx, F. Nat. Mater. 2003, 2, 577-585.
9) Naik, R. R.; Stringer, S. J.; Agarwal, G.; Jones, S. E.; Stone, M. O, Nat. Mater. 2002, 1, 169-172.
10) J. Kim; Y. Rheem; B. Yoo; Y. Chong; K. N. Bozhilov; D. Kim; M. J. Sadowsky; H.-G. Hur; Myung, N. V. Acta Biomater. 2010, 6, 2681-2689.
11) Kim, J.; Myung, N. V.; Hur, H.-G. Chem. Commun. 2010, 46, 4366-4368.
12) Cai, C.; Zhang, L.; Lin, J.; Wang, L. J. Phys. Chem. B 2008, 112, 12666-12673.
13) Karg, M.; Lu, Y.; Carbo-Argibay, E.; Pastoriza-Santos, I.; Perez-Juste, J.; Liz-Marzan, L. M.; Hellweg, T. Langmuir 2009, 25, 3163-3167.
14) Contreras-C, R.; Pacifico, J.; Pastoriza-Santos, I.; P, J.; Fern, A.; Liz-Marz, L. M. Adv. Funct. Mater. 2009, 19, 3070-3076.
15) Fujita, Y.; Mie, M.; Kobatake, E. Biomaterials 2009, 30, 3450-3457.
16) Chen, C.-L.; Rosi, N. L. Angew. Chem.-Int. Edit. 2010, 49, 1924-1942.
17) Zhao, X.; Zhang, S. Chem. Soc. Rev. 2006, 35, 1105-1110.
18) Fu, X.; Wang, Y.; Huang, L.; Sha, Y.; Gui, L.; Lai, L.; Tang, Y. Adv. Mater. 2003, 15, 902-906.
19) Yan, X.; Cui, Y.; He, Q.; Wang, K.; Li, J. Chem. Mater. 2008, 20, 1522-1526.
20) Euliss, L. E.; Grancharov, S. G.; O'Brien, S.; Deming, T. J.; Stucky, G. D.; Murray, C. B.; Held, G. A. Nano Lett. 2003, 3, 1489-1493.
21) Yan, X.; Cui, Y.; Qi, W.; Su, Y.; Yang, Y.; He, Q.; Li, J. Small 2008, 4, 1687-1693.
22) Chen, C.-L.; Zhang, P.; Rosi, N. L. J. Am. Chem. Soc. 2008, 130, 13555-13557.
23) Yan, X.; Zhu, P.; Fei, J.; Li, J. Adv. Mater. 2010, 22, 1283-1287.
24) Reches, M.; Gazit, E. Science 2003, 300, 625-627.
25) Reches, M.; Gazit, E. Curr. Nanosci. 2006, 2, 105-111.
26) Gazit, E. FEBS J. 2007, 274, 317-322.
27) Alfonso, I.; Bru, M.; Burguete, M. I.; Garcia-Verdugo, E.; Luis, S. V. Chem.-Eur. J. 2010, 16, 1246-1255.
28) Liu, L.; Xu, K.; Wang, H.; P. K., J. T.; Fan, W.; Venkatraman, S. S.; Li, L.; Yang, Y.-Y. Nat. Nanotechnol. 2009, 4, 457-463.
29) Yan, X.; He, Q.; Wang, K.; Duan, L.; Cui, Y.; Li, J. Angew. Chem.-Int. Edit. 2007, 46, 2431-2434.
30) Reches, M.; Gazit, E. Nano Lett. 2004, 4, 581-585.
31) Ray, S.; Das, A. K.; Banerjee, A. Chem. Commun. 2006, 26, 2816-2818.
32) Slocik, J. M.; Stone, M. O.; Naik, R. R. Small 2005, 1, 1048-1052.

What is claimed is:

1. A synthesis of peptide-gold nanoparticle hybrid spheres (structures) comprising the step of contacting (i) a gold ion reductant and a gold-binding peptide as a self-assembly inducer with (ii) a gold salt to induce self-assembly of the gold-binding peptide, and forming a scaffold to a hybrid structure as soon as forming a gold nanoparticle in the scaffold.

2. The synthesis of claim 1, wherein the gold-binding peptide comprises an amino acid having an aromatic functional group as R-group.

3. The synthesis of claim 2, wherein the gold-binding peptide comprises at least 2 amino acids having an aromatic functional group as R-group.

4. The synthesis of claim 3, wherein the gold-binding peptide consist of 10-15 amino acids.

5. The synthesis of claim 4, wherein a peptide of the gold-binding peptide comprises the amino acid sequence selected from the group consisting of SEQ ID NOs: 1 and 2.

6. The synthesis of claim 1, wherein the gold salt is selected from a group consisting of HAuCl₄, HAuBr₄, NaAuCl₄, AuCl₃.3H₂O and NaAuCl₄.2H₂O.

7. The synthesis of claim 1, wherein the synthesis is conducted in an aqueous solution condition.

8. The synthesis of claim 7, wherein the aqueous solution condition comprises a condition of pH 1-5.

9. The synthesis of claim 8, wherein the aqueous solution condition comprises a condition of pH 3-4.

10. The synthesis of claim 1, wherein size of the peptide-gold nanoparticle hybrid spheres is changed according to temperature.

11. The synthesis of claim 10, wherein size of the peptide-gold nanoparticle hybrid spheres becomes small with increased temperature.

12. The synthesis of claim 1, wherein distribution of the peptide-gold nanoparticle hybrid spheres becomes consistent with increased temperature.

13. The synthesis of claim 1, which further comprises the step of functionalizing the surface of the peptide-gold nanoparticle hybrid spheres.

14. The synthesis of claim 13, wherein the functionalization is conducted by using a marker.

15. Peptide-gold nanoparticle hybrid spheres produced by a method according to claim 1.

16. A marker composition which comprises the peptide-gold nanoparticle hybrid spheres of claim 15, and the surfaces of the peptide-gold nanoparticle hybrid spheres are functionalized with marker.
